(19) United States
(12) Patent Application Publication
(10) Pub. No.: US 2010/0175147 A1
(43) Pub. Date: Jul. 8, 2010

(54) Functional expression of higher plant nitrate transporters in Pichia Pastoris

(75) Inventors: Liu Lu, Palo Alto, CA (US); Hoa Giang, Oakland, CA (US); Dale F. Loussaert, Clive, IA (US); Haiyin Wang, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: PIONEER HI-BRED INTERNATIONAL, INC., Johnston, IA (US)

(21) Appl. No.: 12/726,369

(22) Filed: Mar. 18, 2010

Related U.S. Application Data
(63) Continuation of application No. 12/136,173, filed on Jun. 10, 2008.
(60) Provisional application No. 60/944,223, filed on Jun. 15, 2007.

(51) Int. Cl. A01H 1/00; C12Q 1/02; C12N 1/19

(52) U.S. Cl. 800/278; 435/29; 435/254.11

(57) ABSTRACT

The present invention relates to a system for functional expression of higher plant nitrate transporter (Nrt) genes in Pichia pastoris, an in vivo nitrate uptake assay using these Pichia pastoris transformants and an assay for readily identifying successful transformants.

### CROSS REFERENCE

This utility application is a continuation of U.S. Utility patent application Ser. No. 12/136,173 filed on Jun. 10, 2008 which claims the benefit U.S. Provisional Patent Application Ser. No. 60/944,223, filed Jun. 15, 2007, both of which are incorporated herein by reference.

### FIELD OF THE INVENTION

The present invention relates to the field of plant molecular biology, more particularly to higher plant nitrate transporters.

### BACKGROUND OF THE INVENTION

Higher plants are autotrophic organisms that can synthesize all of their molecular components from inorganic nutrients obtained from the local environment. Nitrogen is a key element in many compounds present in plant cells. It is found in the nucleoside phosphates and amino acids that form the building blocks of nucleic acids and proteins, respectively. Availability of nitrogen for crop plants is an important limiting factor in agricultural production and the importance of nitrogen is demonstrated by the fact that only oxygen, carbon and hydrogen are more abundant in higher plant cells. Nitrogen present in the form of ammonia or nitrate is readily absorbed and assimilated by higher plants.

Nitrate is the principal source of nitrogen that is available to higher plants under normal field conditions. Thus, the nitrate assimilation pathway is the major point of entry of inorganic nitrogen into organic compounds (Hewitt, et al., (1976) Plant Biochemistry pp 633-6812, Bonner and Varner, eds. Academic Press, NY). Although nitrate is generally the major form of nitrogen available to plants, some plants directly utilize ammonia, mostly under anaerobic conditions.

Nitrate transporter proteins are a member of the Major Facilitator Superfamily (MFS) of proteins. See, Pao, et al., (1998) Microbio. and Mol. Bio. Rev. 62:1-34. Other MFS members include Sugar Porters, Drug:H⁺ Antiporters (14-spanner), Drug:H⁺ Antiporters (12-spanner), Organophosphate:Inorganic Phosphate Antiporters, Oligosaccharide:H⁺ Symporters, Metabolite:H⁺ Symporters, Fucose-Galactose-Glucose:H⁺ Symporters, Phosphate H⁺ Symporters, Nucleoside:H⁺ Symporters, Oxalate:Formate Antiporters, Sialate:H⁺ Symporters, Monocarboxylate Porters, Anion:Cation Symporters, Aromatic Acid:H⁺ Symporters, Unknown Major Facilitators and Cyanate Permeases.

The nitrate uptake system of higher plants consists of a constitutive, low affinity transport system (LATS), which is possibly a carrier system or an anion channel and an inducible or constitutive high affinity transport system (HATS). These systems are regulated by cellular energy supply and intracellular nitrate consumption, and their activity depends on the proton electrochemical gradient. The HATS system is regarded as an H⁺/anion co-transport carrier mechanism that produces transient plasma membrane depolarization upon addition of nitrate. The depolarization is counteracted by the plasma membrane H⁺-ATPase (Ullrich, 1992). The plasma membrane proton ATP-ase is induced by nitrate (Santi, et al., 1995). In plants, the relative activity of the two systems depends on the level of nitrate available in the soil, which can vary greatly (up to four orders of magnitude).

Both LATS and HATS have multiple family members. The NRT1 family is typically a low affinity transport system, while the NRT2 family is typically a high affinity transport system. While most organisms have only a few NRT1 genes (six in humans, four in C. elegans, three in Drosophila and one in yeast), higher plants have large numbers of putative NRT1 genes. For example, Arabidopsis has more than 50 putative NRT1 genes and rice 80. As more genomic information is obtained for additional plants, these numbers only increase.

There are two types of HATS, inducible HATS and constitutive HATS, with the inducible HATS having a much higher rate of transport than the constitutive HATS. Arabidopsis has at least 7 putative HATS and there is some evidence of interaction between the HATS and LATS transport systems. See, Tsay, et al., (2007) FEBS 581:2290-2300.

Two gene products encoding a NRT2-type transporter and an associate membrane protein CrNar2 were identified to be essential for a functional high-affinity nitrate transporter system in Chlamydomonas by X. oocytes. See, Zhou, et al., (2000) FEBS Lett. 466:225-27. Recently, more two-component high-affinity nitrate uptake systems have been identified in higher plants. See, Tong, et al., (2005) Plant J. 41:442-50; Orsel, et al., (2006) Plant Physiology 142:1304-17). A specific interaction between Nrt2 protein and its associate Nar2 protein were identified.

Some nitrate transporter proteins and genes have been isolated and characterized. While the sequences of some genes encoding nitrate transporter proteins are known, as noted above, most nitrate transporter genes are merely putative sequences based on sequence homology compared to known nitrate transporter genes. However, there is a high degree of similarity between nitrate transporter genes and genes encoding other membrane transporter proteins, particularly transporter systems for dipeptides and oligopeptides, so it is unclear how many of these putative genes actually encode nitrate transporters. This is particularly problematic in higher plants, where there are many more putative nitrate transporter proteins than in other types of organisms.

Nitrate transporter proteins contain up to 12 transmembrane domains and are notoriously difficult to characterize biochemically. Functional verification of these sequences is complicated by the fact that currently the only heterologous host for tracking expression and efficacy of such transporters is largely unsatisfactory. The current method of choice for such expression is through expression via Xenopus oocytes. (Miller and Zhou, 1999). This system is widely used for characterization of transmembrane transporter systems that generate electrophysiology changes upon uptake of relevant molecules. The signal-to-noise ratio of the Xenopus system is relatively low, on the order of 2-10 fold, because of the small amount of endogenous transport activity in the oocyte plasma membrane; sometimes nitrate transporter activity is credited with a signal-to-noise ratio of only about twofold.

This method is subject to several problems, such as the short life of the oocytes (typically 2-3 weeks), expense, and the necessity of special equipment. Further, the Xenopus system is also bound with significant variations on cRNA stability, protein translation and protein stability. Also, because each cRNA injection produces a unique event, precise measurement of nitrate uptake kinetics is not possible. Another limitation on the Xenopus system in the context of higher plant nitrate transporter proteins is that the nitrate transported into the Xenopus oocyte via the heterologous nitrate transporter protein produced therein is not readily assimilated or translocated as it is when transported in the actual plant. As a result, it is possible that uptake kinetics determined via the Xenopus system are quite different than the actual kinetic behavior of these proteins in the plants in which they are naturally produced. Further, the Xenopus system is time consuming to test each putative gene for activity.

Pichia pastoris is a non-nitrate assimilating yeast, meaning it does not naturally have nitrate transporter and reduction machinery. Although no such functionality exists naturally, functional expression of nitrate reductase has been achieved, indicating the presence of essential molybdenum cofactor. See, Su, et al., (1997) Plant Physiol. 111:1135-43. More recently, functional co-expression of higher plant nitrate reductase and fugal nitrate transporter has also been achieved. See, Unkles, et al., (2004) J. Bio. Chem. 279:28182. However, there has yet been successful functional expression of higher plant nitrate transporter genes in Pichia pastoris (or any other heterologous system) apart from the Xenopus system described previously.

Identification and verification of the ever-increasing number of putative nitrate transporter genes is important, as once a functional nitrate transporter gene is identified, only then can efforts be efficiently made toward studying the localization of the encoded transporter protein, expression profile and impact on downregulation of its expression on plant growth and development. Accordingly, there is a need for a more efficient and less time-consuming way to verify putative nitrate transporter genes.

In addition, functional expression of higher plant nitrate transporter genes in Pichia pastoris will permit detailed characterization of kinetic behaviors of various nitrate transporter proteins, as a Pichia pastoris system is not hampered by many of the limitations described in the Xenopus system. Detailed characterization of the kinetic behavior of nitrate transporter proteins will make it possible to better classify the various nitrate transporter protein family members. Also, a more complete understanding of the kinetics of these proteins could permit the design of a more efficient nitrate transporter system based on more detailed information from various related plant species, which could then be engineered into, for example, crop plants to give the plants the ability to more efficiently use nitrogen in the soil. These plants would offer better world food security, as well as reduce the negative environmental impact from overuse of nitrogen fertilizers.

### BRIEF SUMMARY OF THE INVENTION

The present invention therefore relates to a system for functional expression of higher plant single-component and two-component nitrate transporter (Nrt) systems in Pichia pastoris, which, in preferred embodiments, eliminates one or more of the disadvantages of the prior art methods described.

The present invention further relates to an in vivo nitrate uptake assay.

The present invention also relates to an assay for identifying successful transformants of a nitrate transporter gene from a putative Nrt gene or library-based screening.

Additional detail regarding preferred embodiments of the present invention will become evident from the further description provided.

### BRIEF DESCRIPTION OF THE FIGURES

FIG. 1 is the results of a Western Blot analysis performed to verify expression of a higher plant nitrate transporter protein, specifically AtNrt1.1.

FIG. 2 is a schematic presentation of P. pastoris strain GS115 carrying various expression cassette(s) of barley HATS. A: GS115 wild type strain, B: GS115-YNR1, C: GS115-Nrt2.1/YNR1, D: GS115-Nar2.3/YNR1, E: GS115-Nrt2.1/Nar2.3-YNR1.

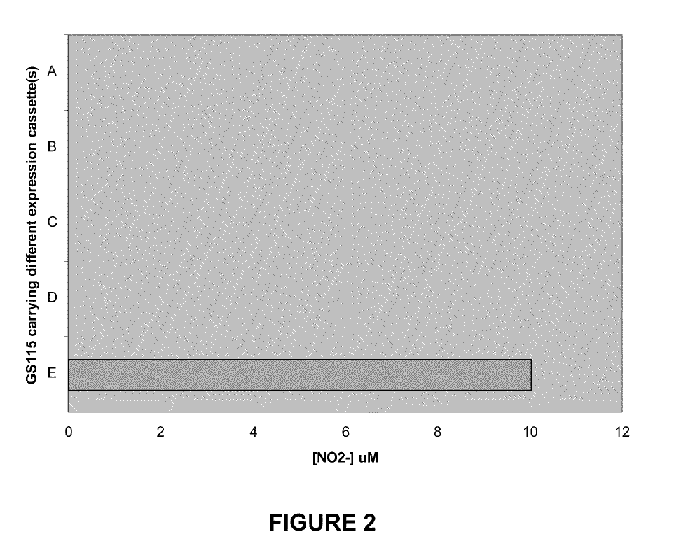

### DETAILED DESCRIPTION

In accordance with the claims, the inventors herein disclose a novel heterologous system for expression of higher plant nitrogen transporter (Nrt) genes. The system generally comprises inserting a gene encoding a plant nitrate transporter protein (or a gene encoding a putative plant nitrate transporter protein) into Pichia pastoris. The Nrt gene is associated with a promoter sequence which permits expression of the Nrt gene in concentrations appropriate to permit the Nrt protein to be functionally expressed in the Pichia pastoris cells. In one embodiment, the promoter is the pAOX promoter (SEQ ID NO: 1). In another embodiment, the promoter sequence used is a constitutive promoter. In further embodiment, the promoter is the pGAP promoter (SEQ ID NO: 2). In another embodiment, the promoter is the pYPT1 promoter (SEQ ID NO: 3).

In another aspect, transformed Pichia pastoris cells are used to assess the kinetics of a nitrate transporter protein of interest. In a further embodiment of the invention, functional transformants are identified by their growth sensitivity to chlorate, making identification of transformants relatively easy and inexpensive.

### Definitions

By “Nrt gene” or “nitrogen transporter gene” is intended one or more nucleotide sequences that encode a protein facilitating nitrogen transport into cells.

By “Nrt” or “nitrogen transporter protein” is intended one or more proteins facilitating nitrogen transport into cells.

By “Nr gene” or “nitrate reductase gene” is intended one or more nucleotide sequences that encode a protein that reduces nitrate to nitrite in cells.

By “Nr” or “nitrate reductase” is intended one or more proteins that reduce nitrate to nitrite in cells.

By “regulatory element” is intended sequences responsible expression of the associated coding sequence including, but not limited to, promoters, terminators, enhancers, introns and the like.

By “promoter” is intended a regulatory region of DNA capable of regulating the transcription of a sequence linked thereto. It usually comprises a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate and further include elements which impact spatial and temporal expression of the linked nucleotide sequence.

### Expression of Nr Proteins

In order to functionally express Nrt proteins, Pichia pastoris must also functionally express a nitrate reductase protein. See, Unkles, et al., (2004) J. Bio. Chem. 279:28182. Accordingly, in order to produce Pichia pastoris transformants capable of nitrate uptake, both an Nrt and Nr gene must be inserted. Any suitable Nr gene may be used, but a preferred Nr gene is the Pichia angusta nitrate reductase (YNR1, SEQ ID NO: 4). Further, multiple Nr genes may also be used.

One acceptable method to obtain Pichia pastoris cells that express a nitrate reductase protein is to use a modified commercially available Pichia pastoris expression vector such as pPICZA, commercially available from Invitrogen. As constitutive promoters are preferred, a portion of the pAOX1 promoter in the pPICZA vector is preferably replaced with a pGAP promoter sequence. Such a sequence may be isolated via Pichia pastoris genomic PCR flanked with Hind III and Xho I restriction sites at both 5′ and 3′ ends. The large vector fragment of Hind III/XhoI digested pPICZA can be used to ligate with the pGAP fragment, and the resulting pPICZA-pGAP vector can be used to clone an Nr gene of choice under control of the pGAP promoter. Other different promoters will also work, however constitutative Pichia promoters are preferred, as they yield more consistent results.

The desired Nr gene is then added to the vector. This will be accomplished differently depending on the particular Nr gene used. For example, if the preferred YNR1 Nr gene is used, it may be cloned into the vector via EcoR I/Not I restriction sites after removal of the EcoR I site at the 3′-end portion of the YNR1 gene, resulting in the pPICZA-pGAP-YNR1 vector. Other acceptable methods also may be used. The resulting vector may then be incorporated into an appropriate Pichia pastoris cell line, such as KM71 or GS115, both available commercially from Invitrogen. After linearization of the vector via either Pme I or Sac I restriction sites, the vector can be integrated into the pAOX1 locus in the Pichia pastoris genome and stable transformants can be identified via the Zeocin antibiotic selection.

### Verification of Nr Expression

A nitrate reductase assay was performed using lysates of the transformed Pichia pastoris cells as a way to assess the functional expression of the transformed Nr genes. See, e.g., Orihuel-Iranzo, et al., (1980) Plant Physiol. 65:595-99; Hageman, et al., (1971) Methods Enzymol. 23:491-503. The transformant with the highest reductase activity was selected as the host for Nrt expression construct transformation.

### Expression of Nrt Proteins

A nitrogen transporter gene (or putative transporter gene) of interest is also incorporated into a vector for insertion into the Pichia pastoris. Of course, the Nrt gene may be equally effectively inserted into the yeast before the Nr gene; the order is simply a matter of preference. A preferred method of producing the Nrt vector is to take a pGAP expression cassette (produced, for example, via Sac I/BamH I restriction sites), and incorporate the pGAP promoter into a vector. As noted previously, the pGAP promoter is preferred, but other promoters are also acceptable, particularly other constitutive promoters. Preferred vectors include those that comprise, in part, the pAOX1 promoter, and most preferred is the pPIC3.5 vector, commercially available from Invitrogen Corporation. The pGAP promoter cassette replaces a major portion of the 5′-AOX promoter via Sac I and Bam HI restriction sites, thereby producing a vector incorporating the pGAP promoter. By way of example, higher plant Nrt genes may be cloned into the vector with promoter (such as pGAP as described above) using BamH I/Not I or Bgl II/Not I restriction sites. The resultant vectors therefore incorporate a suitable promoter and the Nrt gene of interest. The Nrt gene is then incorporated into the previously transformed cells of the Nr expression vector. In a preferred embodiment, the cells are transformed via integration into a locus that will enable selective identification of the successful transformants. In a more preferred embodiment, the Nrt vector is integrated into the His4 gene locus on the Pichia pastoris genome, in which the endogenous His4 gene is not functional, and the transformants selected for on His⁻ medium via complementation of newly-restored functional His4 gene carried from the pPIC3.5 backbone vector.

The disclosed system also may be used with two-component high-affinity nitrate transporter systems (HATS). See, Glass, et al., (2002) J. Experimental Botany 370:855-64. Two-component high-affinity nitrate transporter systems typically contain one gene encoding a typical carrier-type protein Nrt2, and another gene encoding an associate protein Nar2, identified using the Xenopus system noted previously in barley, see, Plant J. (2005) 41:442-50 and in Arabidopsis see, Plant Physiology (2006) 142:1304-17. A highly specific interaction between Nrt2 and Nar2 is required for a functional nitrate transporter system. The specific interaction between HvNrt2.1 and HvNar2.3 cannot be replaced by another closely related gene HvNar2.x or HvNrt2.x in barley. The two membrane components, AtNrt2.1 and AtNar2.1, interact at the protein level. See, Plant Physiology (2006) 142:1304-1317.

These systems are integrated in much the same way, except that both genes for the two components are introduced into the Pichia pastoris cells. They may be introduced via separate vectors, or via the same vector. Preferably, the two genes are introduced with the same vector, as functionality of the transformants appears to increase.

### Verification of Nrt Expression

Confirmation of Nrt expression after transformation may be made in many ways, such as, for example, by tagging the Nrt gene introduced with a marker. Protein markers are preferred. The most preferred marker protein is influenza hemagglutinin (HA), but other markers may also be used. Detection of the marker depends on which marker was used. For example, if HA is used, then Western blot analysis would be an appropriate method of detection. If a fluorescent protein marker is used, the detection would be accomplished visually. Other markers and detection methods are known in the art, and the particular method of detection is not intended to be limiting. Further, it is not necessary to confirm that the Nrt is actually being expressed, although it is desirable to do so in order to ensure further tests are not conducted with unsatisfactory transformants.

A particularly effective way to screen for functional transformants is the use of a chlorate assay. It is known that Pichia pastoris cells with co-expressed Arabidopsis nitrate reductase and a yeast nitrate transporter driven under methanol-inducible AOX promoter are sensitive to 150 mM chlorate on solid medium. See, Unkles, et al., (2004) J. Biol. Chem., 279:28182-86. However, when a constitutive promoter rather than the commonly-used pAOX is used, the sensitivity of the functional transformants to chlorate increases substantially, and therefore the chlorate assay is much more sensitive. For example, when the preferred pGAP promoter is used, functional transformants are sensitive to chlorate concentrations much below 150 mM, such as by concentrations of as low as about 0.5 mM. Of course, the sensitivity level will vary depending on the rate of uptake of the particular Nrt integrated into the Pichia pastoris.

### In Vivo Nitrate Uptake Assay

Pichia pastoris is a non-nitrate assimilating yeast. Because of this, any nitrate taken in via the Nrt is converted to nitrite by the co-expressed Nr and diffused or transported to the medium. This property permits the transformed Pichia pastoris to be used as an in vivo nitrate uptake assay, as the amount of nitrate taken into the cells is directly proportional to the nitrite later present in the growth medium. Nitrite may be quantitatively detected using Griess reagent or any other method known in the art. By way of the assay, various kinetic parameters of an Nrt may be quantified, such as Kₘ and Vₘₐₓ.

The assay is generally performed by incubating transformed Pichia pastoris on a medium containing nitrate for a set amount of time and assessing the amount of nitrite diffused to the medium after that set amount of time. The medium is preferably buffered. Most preferably, the medium has about 20 mM MOPS, pH 6.5, buffer and about 1% glucose, as this buffered medium allows the detection of linear uptake for up to several hours. The table below provides kinetic data for several Nrt proteins ascertained using the in vivo uptake assay herein described:

TABLE 1

| | GenBank | | **Vₘₐₓ |
|---|---|---|---|
| Gene name | accession # | *Kₘ, mM | (μM/m/50 μl cell) |
| AtNrt1.1 | L10357 | 0.36 | 8.0 |
| AtNrt1.3 | AB019232 | 7.4 | 14.4 |
| AtNrt1.4 | AC003105 | 0.61 | 4.0 |
| BnNrt1.1 | AJ278966 | 0.24 | 0.80 |
| NtNrt1.1 | AB102806 | 0.70 | 8.0 |

*Km, mM for Nitrate
**Vmax, (μM/m/50 μl cell), μM of nitrate taken up in one minute by 50 μl Pichia pastoris cell expressing the indicated nitrate transporter gene.

Further, many currently-identified Nrt genes are only classified as such based on sequence homology, and thus are putative Nrt genes. However, due to the high level of similarity between Nrt genes and the genes for other transporter proteins, many of these putative Nrt genes do not actually encode Nrt proteins. Accordingly, the in vivo nitrate uptake assay herein described is a comparatively inexpensive way to determine whether such putative genes do, in fact, actually encode an Nrt protein. Similarly, the assay may be used to verify Nr activity, as Pichia pastoris will not uptake nitrate without an appropriate reductase expressed, especially when the nitrate concentration is less than 2 mM.

### EXAMPLES

### Example 1

### Co-Expression of Pichia angusta Nitrate Reductase and Higher Plant Nitrogen Transporter Genes

In order to produce Pichia pastoris transformants, PCR was used to obtain the pGAP promoter cassette from Pichia pastoris genomic DNA via Hind IIII and Xho I restriction sites at the respective 5′ and 3′ ends. The pGAP promoter sequence is SEQ ID NO: 2. A partial DNA sequence of the 5′-AOX1 promoter of the pPICZA vector (commercially available from Invitrogen) was replaced with the pGAP promoter cassette using Hind III and Xho I to generate pPICZA-pGAP. YNR1 (SEQ ID NO: 4) was then cloned via EcoR I/Not I sites into the pPICZA-pGAP construct to form pPICZA-pGAP-pYNR1. This was then transformed into a commercially available strain of Pichia pastoris, KM71, also available from Invitrogen. The success of the transformation was verified by lysing the cells and assessing the nitrate reductase activity of the lysate. The nitrate reductase activity of the lysate was assessed using NADH and nitrate as a substrate and quantified by Griess reagent. See, Cortas and Wakid, (1990) Clin. Chem. 36:1440-43.

Clone 5 (KM71-YNR1) was selected as the host strain for co-transformation of higher plant Nrt genes. Introduction of the higher plant genes proceeded in similar fashion. A pGAP promoter cassette (Sac I/BamH I) was inserted in place of a portion of the 5′-AOX promoter of the pPIC3.5 vector, commercially available from Invitrogen. This generated a pPIC3.5-pGAP vector construct. Various higher plant genes were then cloned as BamH I/Not I or Bgl II/Not I cassettes to generate pPIC3.5-pGAP-Nrt vectors. The Nrt genes transformed include AtNrt1.1, AtNrt1.3, AtNrt1.4, BnNrt1.1 and NtNrt1.1. The pPIC3.5-pGAP-Nrt vectors were then transformed into the Pichia pastoris strain via integration into the His4 gene locus. Transformants were then selected on His⁻ solid medium. These transformants express both the YNR1 nitrate reductase as well as one of the various higher plant Nrt genes.

Transformation was further confirmed by Western blot analysis. The Nrt genes used were tagged with an influenza hemagglutinin epitope, HA, at their c-termini. HA is commonly used to detect protein expression in various systems, including yeast. Pichia pastoris cell pellets expressing Nrt genes from 1.5 mL of overnight YPD cultures were resuspended in 0.5 mL of extraction buffer comprising 250 mM sucrose, 5% v/v glycerol, 1 mM magnesium chloride, 1 mM EDTA, 25 mM MOPS, 1 mM DTT, 100 μM PMSF and a proper amount of complete mini protease inhibitor mixture, pH 7.2, with 0.4 g cold sand. A Geno-grinder, available from Spex CertiPrep, was used to break the yeast cells. The suspensions after treatment were centrifuged for 10 minutes at 5000 rpm to remove the sand and cell debris.

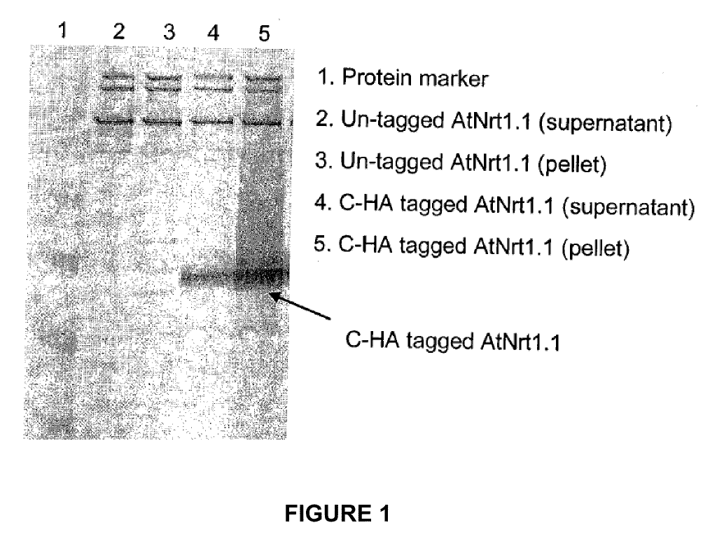

The supernatants were then further centrifuged for 45 minutes at 30,000 rpm. The gelatinous pellets (membrane fraction) were resuspended in the extraction buffers and used for Western blot analysis using standard protocol. Primary antibody (rabbit anti-HA) preparation was obtained from Zymed and secondary antibody (goat anti-rabbit biotinylated) was obtained from Bio-Rad. The result of the Western blot for the AtNrt1.1 transformant is shown in FIG. 1. C-HA tagged AtNrt1.1 protein was detected, therefore confirming the success of the transformation and the expression of the Nrt genes in the transformants.

### Example 2

### Expression of Barley Two-Component HATS

To test if P. pastoris was capable to identify a plant two-component HATS system, the following constructs containing HvNrt2.1 (SEQ ID NO: 5) or HvNar2.3 (SEQ ID NO: 6) were made and transformed into GS115-YNR1 line at AOX locus individually: pPIC3.5-pGAP-HvNrt2.1 or pPIC3.5-pGAP-HvNar2.3. To obtain transformants carrying HvNar2.3 and HvNrt2.1, GS115 wt was transformed with pPIC3.5-pGAPHvNar2.3-pGAPYNR at His 4 locus, then re-transformed with pPIC3.5-pGAP-HvNrt2.1 at AOX locus after the line was confirmed carrying both YNR1 and HvNar2.3 genes by PCR. The transformants were cultured in rich media (YPD) at 30° C. for overnight. Yeast cells were collected and washed with water twice then re-suspended in 20 μM MOPS, pH 6.5 and 1% glucose containing 1 mM NaNO₃. After 2 hours incubation at 30° C., the supernatant was collected for nitrite assay with 1% Sulfanilamide, 0.01% N-(1-Naphthyl)ethylene-diamine dihydrochloride and 15% (v/v) H₃PO₄. The transformants containing either HvNrt2.1 or HvNar2.3 did not have NT activity. However, the transformants co-expressed HvNrt2.1 and HvNar2.3 had NT activity compared to the negative controls (GS115 wt and GS115-YNR1 line). Notably, the Pichia system offered significant advantages for the two-component higher plant HATS over the Xenopus system due to its increased signal to noise level. When tested in Xenopus, the same barley two component HATS only showed about 4-fold signal to noise ratio, see, Plant J. (2005) 41:442-50, while, in the Pichia system, the background noise is not detectable among the physiologically relevant nitrate concentration range (under 2 mM).

### Example 3

### In Vivo Nitrate Uptake Assay

Pichia pastoris transformants were prepared in accordance with example 1 above and grown in YPD medium at 30° C. for 1 day. Cell pellets were washed twice with water then resuspended with a proper amount of uptake medium including nitrate. 24 different nitrate concentrations were tested, ranging from 0-30 mM. Optimal results were obtained with a buffered medium such as 20 mM MOPS, pH 6.5, 1% glucose, and the varying concentrations of nitrate. The aliquots were transferred into a 96-well plate; supernatants were mixed with Greiss reagent and monitored at 545 nm using a plate reader produced by Molecular Devices.

The raw data were fitted into proper kinetic equations, such as the Michaelis-Menton equation, using a program such as KaleidaGraph, available from Synergy Software, Reading Pa., USA. This in vivo uptake assay permits the kinetics of various nitrate transporters to be ascertained, such as those listed in Table 1 above.

### Example 4

### Functional Pre-Screening Assay

5 μL of overnight YPD transformant cultures were spotted onto a minimal plate containing yeast nitrogen base, 10 mM proline, 2% glucose and 3-20 mM chlorate. These were incubated overnight at 30° C. The amount of growth was observed the next morning. Those cells that were functional transformants were inhibited by the chlorate in the medium and nonfunctional transformants exhibit normal growth. This assay was repeated with various Nrt genes. It was found that Pichia angusta Nrt transformants were inhibited in 0.5 mM chlorate, while other Nrt transformants, such as Arabidopsis Nrt1.1 transformants, were less sensitive to very small concentrations of chlorate.

Although the foregoing has been described in some detail by way of illustration and example for purposes of clarity of understanding, it will be obvious that certain changes and modifications may be practiced within the scope of the appended claims. All references cited herein are incorporated by reference in their entirety.

What is claimed is:

1. A method of heterologously expressing higher plant nitrogen transporter genes in vivo comprising:
incorporating a nitrate reductase gene into a first vector;
using the vector to transform the nitrate reductase gene into a Pichia pastoris cell;
incorporating a higher plant nitrate transporter gene into the first or a second vector; and
using the vector to transform the higher plant nitrate transporter gene into said Pichia pastoris cell.

2. The method of claim 1 wherein the nitrate transporter gene is incorporated into the first vector.

3. The method of claim 1 wherein the nitrate transporter gene is incorporated into a second vector.

4. The method of claim 1 wherein the first vector further comprises a promoter.

5. The method of claim 4 wherein the promoter comprises a constitutive promoter.

6. The method of claim 5 wherein the constitutive promoter is selected from the group consisting of the pGAP promoter and the pYPT1 promoter.

7. The method of claim 3 wherein the second vector further comprises a promoter.

8. The method of claim 7 wherein the promoter comprises a constitutive promoter.

9. The method of claim 8 wherein the constitutive promoter is selected from the group consisting of the pGAP promoter and the pYPT1 promoter.

10. The method of claim 1 wherein the Pichia pastoris cell is selected from the group consisting of a cell from the KM71 cell line and a cell from the GS115 cell line.

11. A method of determining the kinetic properties of one or more nitrate transporter proteins, comprising:
producing a first vector incorporating a first constitutive promoter and a nitrate reductase protein;
transforming the Pichia pastoris cells with the first vector;
producing a second vector incorporating a second constitutive promoter and one or more nitrate transporter proteins;
transforming Pichia pastoris cells with the second vector;
growing the transformed Pichia pastoris cells in two or more growth media, wherein each growth medium comprises a different level of nitrate;
measuring the amount of nitrite produced by the transformed Pichia pastoris cells over a period of time; and
using the measurements to determine the kinetic properties of the nitrate transport protein.

12. The method of claim 11 wherein the first constitutive promoter is selected from the group consisting of the pGAP promoter and the pYPT1 promoter.

13. The method of claim 11 wherein the second constitutive promoter is selected from the group consisting of the pGAP promoter and the pYPT1 promoter.

14. The method of claim 11 wherein the nitrate reductase is YNR1.

15. The method of claim 11 wherein the first vector is pPICZA-pGAP-YNR1.

16. The method of claim 11 wherein the second vector is pPIC3.5-pGAP-Nrt.

17. The method of claim 11 wherein the growth media further comprise a buffer.

18. The method of claim 17 wherein the buffer comprises about 20 mM 3-(N-morpholino)propanesulfonic acid (MOPS).

19. The method of claim 11 wherein the nitrogen transporter protein comprises a two-component high-affinity nitrate transporter system (HATS).

20. The method of claim 19 wherein the second vector is selected from the group containing expression cassettes of pGAP-HvNrt2.1 (SEQ ID NO: 5), pGAP-HvNar2.3 (SEQ ID NO: 6) and/or pGAPHvNar2.3 (SEQ ID NO: 6)-pGAPYNR (SEQ ID NO: 4).

21. A method of determining whether transformed Pichia pastoris cells functionally express a nitrate transporter protein and a nitrate reductase protein, comprising:
placing transformed Pichia pastoris cells onto growth media comprising chlorate, wherein the concentration of chlorate is between about 0.5 mM and about 20 mM;
allowing the cells to grow overnight; and
assessing whether cell growth was inhibited.

22. The method of claim 21 wherein the concentration of chlorate is between about 0.5 mM and about 15 mM.

23. The method of claim 21 wherein the concentration of chlorate is between about 0.5 mM and about 10 mM.

24. The method of claim 21 wherein the concentration of chlorate is between about 0.5 mM and about 5 mM.

25. The method of claim 21 wherein the concentration of chlorate is between about 0.5 mM and about 2 mM.

26. The method of claim 21 wherein the concentration of chlorate is about 0.5 mM.

27. A Pichia pastoris cell transformed in accordance with the method of claim 1.


SEQUENCE LISTING (6 sequences)

SEQ ID NO: 1 - DNA, 1866 bp, Pichia pastoris
ctgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctctctgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagat

SEQ ID NO: 2 - DNA, 487 bp, Pichia pastoris
ttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaagtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 3 - DNA, 509 bp, Pichia pastoris
ccatatgatgagtcacaatctgcttccacagacgagtacaaggacaggcaaaaggaattggaagaagttgctaacccaataatgagcaagttctatggagctgctggtggagctcctggtggagctcctggtggcttccctggaggtttccctggcggagctggcgcagctggcggtgccccaggtggtgctgccccaggcggagacagcggaccaaccgtggaagaagtcgattaagcaattcaacggataaattctggttaatatatataacgtgaataggaaattaaggaaattttggatctaataatgtgctgtatgccgacatcgggcatcgtagattgtatagtatcgctgacactataataagccagccaaaacccctaaaccagttgccctccactaattagtgtactacccaatcttgcctcttcgggtgtcttttataaggacagattcacaagctcttgttgcccaatacacacatacacacagagataatagcagtc

SEQ ID NO: 4 - DNA, 2580 bp, Pichia angusta
atggattctattgtcactgaggtaacctatggtctggagatcaagaaaatcaaagatatcacggagctaccttttccagtcaggcaagactctcctcttaccgaggtgcttccaacagatctgaagaccaaagataattttgtcgctagagatcctgaccttcttagactcactggttcacacccattcaattctgagccgccactgacaaagctttatgactcggggtttctcactccagtgagtcttcactttgtgagaaaccacggccccgttccttacgttcctgatgaaaatattttagactgggaagtttcaattgaagggatggttgaaacgccttataaaatcaaattgtcagacataatggagcagtttgatatctattcaacccccgttactatggtctgcgctggaaacagaagaaaggagcagaatatggtaaagaagggagccggtttcaattggggagcagctggaacatctacttctctttggacaggatgcatgcttggagatgtaataggcaaggctagaccatcaaagagagcaagattcgtatggatggagggtgcagataatccggcaaacggcgcataccgcacctgtatccgcttaagctggtgtatggaccctgaacggtgcatcatgatcgcataccagcagaacggcgagtggttgcatcctgaccatggaaagccccttcgagtagtaatccccggtgttattggtggacgatcagtcaaatggctaaagaaactagtagtgagcgatcggccgtctgaaaattggtatcattattttgataatcgggttcttccaaccatggtgacgccagagatggctaaaagtgacgacaggtggtggaaagacgagcgatatgccatatatgatctgaacttgcaaacgatcatttgcaagcccgaaaatcagcaggttatcaagatttcagaggacgagtacgaaattgcaggttttggctacaacggaggtggagtcagaataggccggattgaggtcagtcttgacaaagggaagagttggaaactggcagatatagactatccggaagacagatatagggaagcaggttacttcagattgtttggcggacttgtgaatgtttgcgacagaatgagctgcctgtgctggtgtttctggaagctcaaggttcctctttctgaattagcaaggtcaaaagatattctcattcgtggcatggatgagcgtatgatggttcagccgcgcacgatgtactggaacgtaacgtccatgctgaacaactggtggtatcgagtcgccattatccgcgagggtgagagtcttcgatttgagcatcccgtggtggccaacaagcctggcggttggatggatagggtcaaggcagagggtggagatattctagataacaattggggagaggtggacgacacggtcaagcaggctgaaaggaggccgcatattgatgaggatctcgagatgatgtgcaaccgggagaaaatggacgtcgttatcaaatattcagagtttgaagcacacaaggacagcgagacagagccatggtttgctgtcaaaggccaagtgttcgatggtagctcgtatctggaagaccatccaggaggggcccagtcgatcttgatggtgagcggcgaagacgcaaccgacgacttcattgcaattcactcatcttttgctaaaaagctgctcccttccatgcacctgggaagactcgaagaggtcagttcagttacaaaagtgaagtctgtagagcaaaatgtgaagcgagaagttttgctcgatccgcgaaaatggcacaagataacgcttgcagaaaaagaggttatctcttccgactctagaatattcaagttcgacctggagcattcagaacagctttctggtctgccaacgggtaaacacctgtttctgaggctaaaagattcatctggcaagtatgtgatgagggcatacacccctaaatcgagcaattctttgcggggtcgtctagagatattgataaaggtttatttcccaaatagggaataccccaacggcggaattatgacaaatcttatcgaaaacctccaagtgggaaaccagatcgaggtcaaaggacctgtcggcgagtttgagtatgtcaagtgcgggcactgcagtttcaataacaagccttatcaaatgaagcattttgttatgatctcgggaggatcgggcatcaccccaacttaccaggttctgcaagctattttcagcgatcccgaagatagaacaagcgttcagttattttttggaaataaaaaagttgacgatatcctgcttcgagaagagcttgaccacatacaggaaaaatatccagaacaattcaaagttgattactcgctgtcggatctggatcatctaccggagaattggagcggagtgagaggcaggttaacattcgatattctagacacctatgttcgggggaaaaaaatgggagagtatatgctactggtatgtggaccgccaggaatgaacggtgtggtcgaaaactggtgcaatgcgcgcaagttggataaacagtatgtagtgtacttctga

SEQ ID NO: 5 - DNA, 1524 bp, Hordeum vulgare
atggaggtcgaggcgggcgcccatggcgacactgccgcgagcaagttcacgctgccggtagactccgagcacaaggccaagtccttcaggctcttctccttcgccaacccgcacatgcgcaccttccatctctcgtggatctccttcttcacttgcttcatctccaccttcgccgcagcgccccttgtccccatcattcgtgataacctcaaccttgccaaggccgacatcggcaatgccggtgtggcatccgtttctgggtccatcttctccaggcttgccatgggtgccatctgcgatctcctcgggccgcggtatggatgtgcattcctcgtcatgctctcggcaccgaccgttttctgcatggccgttatcgatgatgcctcagggtacatcgccgtccgctttctcattggcttctcgcttgctacgttcgtgtcatgccaatattggatgagcaccatgtttaatagcaagatcatcggcacagtcaacggcctcgctgctggatggggcaacatgggtggtggcgccacgcagctcatcatgccgctcgtcttccatgcaatccagaagtgtggtgccacgcccttcgtagcgtggcgtattgcctacttcgtgcccggaatgatgcacatcgtgatgggcttgttggtactcaccatggggcaagatctccctgatgggaacctcgcaagtctccagaagaagggagacatggccaaggacaagttctccaaggtcctttggggcgccgttaccaactaccgaacatggatctttgtcctcctctatggctactgcatgggtgtcgagctcaccaccgacaatgtcattgccgagtactacttcgaccacttccacctagacctccgtgccgccggtaccatcgctgcctgcttcggcatggccaacatcgtcgcacgtcctacgggtggctacctctctgaccttggcgcccgctatttcggcatgcgtgctcgcctctggaatatctggatcctccaaaccgctggtggcgctttctgcatctggctcggtcgtgcatcggccctccctgcctcggtgaccgccatggtcctcttctccatctgcgcccaggctgcgtgtggtgctatctttggtgtcgcacccttcgtctccaggcgttcccttggcatcatttccgggttgaccggtgctggtggaaacgtgggcgcagggctcacacagcttctcttcttcacgtcatcgcaatactccactggtaggggtctcgagtacatgggcatcatgatcatggcatgcacgctgcccgtcgctcttgtgcacttcccacaatggggatccatgttcttccctgccagcgccgacgccacggaggaggagtactacgcctcggagtggtccgaagaggagaaagccaagggtctccatatcgccggccaaaaatttgctgagaattcccgctcggagcgcggtaggcgcaacgtcatccttgccacgtccgccacaccacccaacaatacgccccagcacgtatga

SEQ ID NO: 6 - DNA, 597 bp, Hordeum vulgare
atggctcggcaaggcatggtcacggcgctgctgctgctggtcctcgccgccggctgctgcgcatcggcgggcgccgtggcgtacctctccaagctgcctgtgaccctcgacgtcaccgcctcccccactcccggccaagttcttcacgccggcgaggacgtgatcacggtgacgtgggccctgaacgcgagccagccggccggcaaggacgccgactacaagaacgtgaaggtgagcctctgctacgcgccggtgagccagaaggagcgcgagtggcgcaaaacccacgacgacctcaagaaggacaagacctgccagttcaagatcacccagcaggcctaccccggcgccggcaaggttgagtatcgcgtcgccctcgacatccccaccgccacttactacgtgcgcgcctacgcgctcgacgcctcgggcacccaggtcgcctacggccaaaccgcgccaaccgccgccttcaacgtcgtcagcatcacgggagtcaccacctccatcaaggtcgccgccggcgtcttctccgccttctccgtcgcctccctcgccttcttcttcttcattgagaaacgcaagaagaacaactaa